(19) United States
(12) Patent Application Publication
(10) Pub. No.: US 2023/0169611 A1
(43) Pub. Date: Jun. 1, 2023

(54) METHODS, SYSTEMS, AND COMPUTER READABLE MEDIA FOR UNIFIED PRODUCTION RECIPE MANAGEMENT AND NUTRITION CONTENT CALCULATION

(71) Applicant: Parsley Software Inc., Scotts Valley, CA (US)

(72) Inventor: Hava Volterra, Scotts Valley, CA (US)

(21) Appl. No.: 17/893,775

(22) Filed: Aug. 23, 2022

Related U.S. Application Data
(60) Provisional application No. 63/236,206, filed on Aug. 23, 2021.

(51) Int. Cl. G06Q 50/10; G09B 19/00

(52) U.S. Cl. CPC G06Q 50/10; G09B 19/0092

(57) ABSTRACT

A method for unified production food recipe management and nutrition content calculation includes receiving input from a user regarding production food recipe ingredients and quantities of the ingredients used by the production food recipe. The method further includes receiving input from the user regarding at least one uptake percentage or automatically determining at least one uptake percentage for at least one of the ingredients that will be partially discarded. The method further includes calculating, using the input regarding the production food recipe ingredients and quantities of the ingredients used by the production food recipe and the at least one uptake percentage, nutrition content of a consumable portion of food prepared using the production food recipe. The method further includes outputting the nutrition content of the consumable portion of food prepared using the production food recipe.

### PRIORITY CLAIM

This application claims the benefit of U.S. Provisional Patent Application Ser. No. 63/236,206 filed Aug. 23, 2021, the disclosure of which is incorporated herein by reference in its entirety.

### TECHNICAL FIELD

The subject matter described herein relates to recipe management and nutrition content calculation. More particularly the subject matter described herein relates calculating accurate nutrition facts of brined, marinated, and deep-fried foods on recipe management, and nutrient and dietary analysis systems and software.

### BACKGROUND

As people increasingly want to know the dietary content of their meals, and as menus change more rapidly, the need to obtain recipe nutrition facts automatically becomes increasingly important. In addition, the ability to obtain this information directly from the same recipes used for cooking the dishes, in lieu of having two sets of recipes (one for production purposes, and one for calculating nutrition facts), provides a tremendous savings in time while greatly reducing the errors that can occur if the two sets are not kept entirely in sync.

Recipes used for production can be out of sync with recipes used to calculate nutrition facts when some of the ingredients used for cooking are discarded or otherwise not included in the final cooked or otherwise prepared food product. For example, in the case of brines and marinades, only a small percentage of the brine or marinade is absorbed by the food, with the remainder being discarded. If the brine or marinade contains 100 mg of sodium, for example, and only 15 mg is absorbed, the production recipe would specify 100 mg of sodium, but the nutrition content calculation for the recipe would also indicate 100 mg of sodium, which is inaccurately high. If a separate recipe is used to calculate the nutrition content, it becomes burdensome on the user to maintain two recipes for each dish—one for production and one for calculating nutrition content. A similar problem can occur with foods cooked in fats or oils where a high percentage of the fats or oils is not absorbed by the consumable portion of food prepared using the recipe.

In light of these and other difficulties, there exists a need for improved methods, systems, and non-transitory computer readable media for unified production food recipe management and nutrition content calculation that avoids at least some of these difficulties.

### SUMMARY

A method for unified production food recipe management and nutrition content calculation includes receiving input from a user regarding production food recipe ingredients and quantities of the ingredients used by the production food recipe. The method further includes receiving input from the user regarding at least one uptake percentage or automatically determining at least one uptake percentage for at least one of the ingredients that will be partially discarded. The method further includes calculating, using the input regarding production food recipe ingredients and quantities of the ingredients used by the production food recipe and the at least one uptake percentage, nutrition content of a consumable portion of food prepared using the production recipe. The method further includes outputting the nutrition content of the consumable portion of food prepared using the recipe.

The subject matter described herein can be implemented in software in combination with hardware and/or firmware. For example, the subject matter described herein can be implemented in software executed by a processor. In one exemplary implementation, the subject matter described herein can be implemented using a non-transitory computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

Exemplary implementations of the subject matter described herein will now be explained with reference to the accompanying drawings, of which:

FIG. 1 is block diagram illustrating the formulation of a recipe from sub-recipes;

FIG. 2 is a block diagram illustrating the structure of a unified production recipe usable for both production and nutrition calculation;

FIGS. 3-6 are computer screenshots showing an exemplary implementation of the uptake percentage on unified production food recipe management and nutrition content calculation software;

FIG. 7 is a block diagram of a server implementing unified production food recipe management and nutrition calculation; and

FIG. 8 is a flow chart illustrating an exemplary process for unified production food recipe management and nutrition calculation.

### DETAILED DESCRIPTION

### Standard Structure and Method for Obtaining Nutrition Facts of Recipes in Current Recipe Management and Nutrition Management Systems

Recipe management and nutrient and dietary analysis systems and software traditionally calculate the nutrition facts for a recipe by adding up the nutrition facts for each ingredient in the recipe, multiplying the nutrition facts for an ingredient by the quantity of that ingredient in each portion or serving of the recipe, and adding up those numbers. The recipe management and nutrient and dietary analysis systems and software then determine allergens by marking any allergen contained in any of the ingredients of the recipe.

For purposes of convenience, both in managing recipes and in setting up cooking production plans, recipes used in commercial cooking environments are often sub-divided into sub-recipes, with one top level recipe using the sub-recipes to create the final dish. For example, a sub-recipe could be a spice mix, another sub-recipe could be a salad dressing that uses that spice mix, and the final recipe could be a mixed green salad that uses the salad dressing and mixes it with lettuce greens.

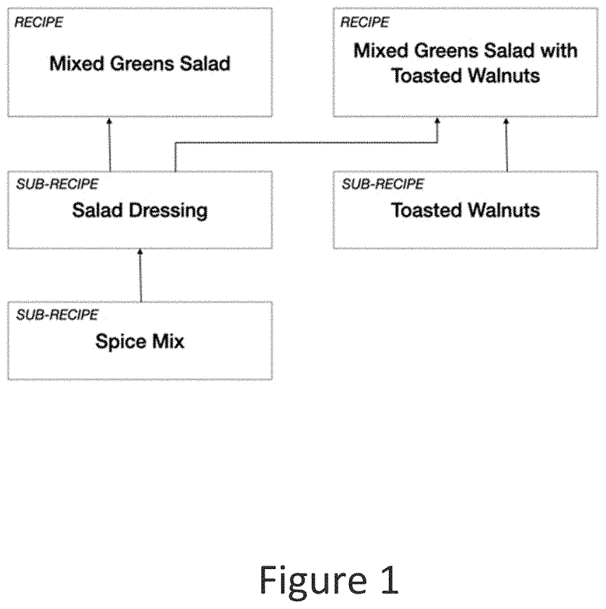

FIG. 1 illustrates an example of sub-recipes used to make two different recipes—one for mixed greens salad and one for mixed greens salad with toasted walnuts. Both recipes use the same sub-recipe called salad dressing, which in turn uses the sub-recipe called spice mix. In addition, the mixed greens salad with toasted walnuts recipe uses an additional sub-recipe called toasted walnuts.

In general, any sub-recipe can use any other sub-recipe, and any recipe can use any sub-recipe. The nutrition facts for a recipe are calculated by adding all the nutrients in the ingredients used in the recipe and in the sub-recipes that this recipe uses and multiplying them by the quantity used for a portion or serving.

### The Problem

When using the method described above to calculate the nutrition facts of a recipe, certain cooking methods can result in unreliable nutrition facts. In particular, these three types of cooking methods can compromise the results;
1. Marinating or pickling can result in calculated nutritional levels (in particular of sodium and sometimes of fat or protein) that are beyond the correct numbers, since much of the marinade is discarded.
2. Cooking in salted water (such as cooking pasta) results in nutritional sodium levels that are significantly above the correct numbers, since much of the cooking water is discarded.
3. Deep frying or including oils and fats in the marinade can result in incorrectly high fat numbers, since most of the oils and fats are discarded or reused.

### Our Solution

We solve this problem by adding a variable to each sub-recipe, which we have called uptake percentage. Any marinade, or brining or pickling liquid, or salt water for boiling ingredients, or oil for deep frying, must be placed by the recipe author in a sub-recipe. For each sub-recipe, the recipe author will be able to enter the uptake percentage. In one example, the uptake percentage may be set by default to 100%, but the author can modify the uptake percentage according to the uptake characteristics of that sub-recipe. For example, a nominal number for brines is 15%, for cooking in salt water 25%, meaning that this is the percentage that ends up in the dish that the customer gets. In another example, the unified production food recipe management and nutrition content calculation software may automatically determine an uptake percentage for a sub-recipe based on the type of ingredient for the sub-recipe based on a heuristically determined value or other algorithm for the uptake percentage. For example, for marinades and meats, it may be determined experimentally that a meat that is properly marinated for taste purposes absorbs 15% of the marinade. In such a case, the unified production food recipe management and nutrition content calculation software may be configured to use 15% as the automatically determined uptake percentage for marinades for meats. The automatically determined uptake percentage value may be presented to and be modifiable by the user. In one example, the nominal uptake percentage for brines may be be set to 6%, for marinades at 15%, for deep frying at 3%, and for salt-water cooked dishes at 5%.

The subject matter described herein is not limited to using a heuristics-determined uptake percentage. In another example, an artificial intelligence-driven uptake percentage may be used. For example, an artificial intelligence or machine learning algorithm may be trained to determine the uptake percentage for sub-recipe ingredients based on ingredient type, cooking method, and other factors, such as brining or marinating time. The trained Al algorithm may be a component of the unified production food recipe management and nutrition content calculation software

The heuristics-driven or Al driven automatic setting of the uptake percentage allows the software to automatically determine the uptake percentage of brine, marinade, and salt water sub-recipes based on the recipes that use them. The automatically determined values may be based on the fact that reasonably cooked or otherwise prepared food (that has what is normally considered by diners to be a palatable sodium quantity) will have, for meats and vegetables, about 1150 mg per pound of raw meat or vegetables, and for dry goods, including rices, pastas, and dry beans, about 2300 mg of sodium per pound of dry goods. For example, if the brine includes 30,000 mg of sodium and is used for 2 pounds of meat, then the uptake percentage will automatically be set as follows:

Uptake Percentage=100*((2*1150)/30,000)=7.67%

An alternative method may be performed where instead of requiring the user to place the ingredients that will be partially discarded (such as the brine or the marinade ingredients) into a sub-recipe, they will be able to leave them in the recipe and mark them as requiring an Uptake Percentage. The system will then use the same heuristic-driven or AI-driven logic to determine the uptake percentage for those ingredients and use it to determine those ingredients' contribution to the nutrition content of the finished dish.

Once you add the uptake percentage, the nutrition facts for any recipes using, for example, brine, will have only 6% of the nutritional value of the brine that was added to the recipe. The recipe that uses it can also be a sub-recipe, for example Pickles could be a sub-recipe used in many dishes, and it could use the sub-recipe called Pickling Brine.

This means that the recipe will be accurate for the cooks that use it, while the uptake percentage ensures that the nutrition facts of the recipe and sub-recipes that use the pickling brine will be correct as well.

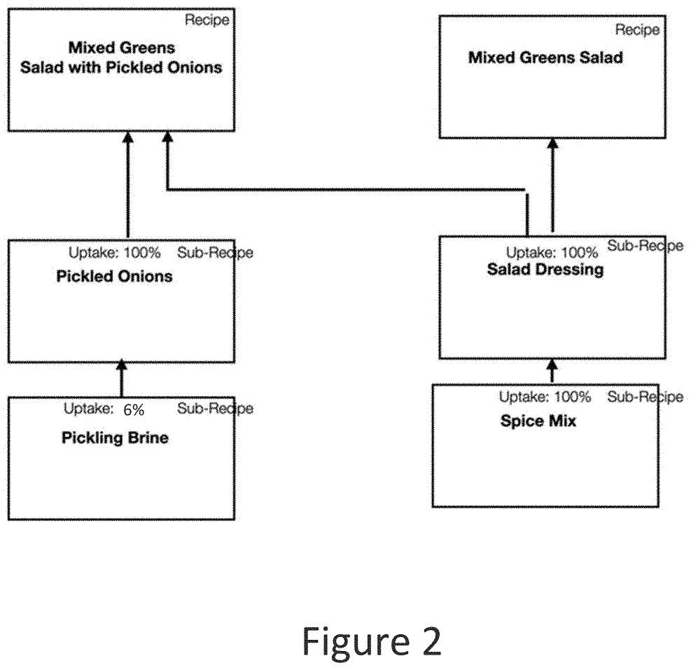

FIG. 2 illustrates a recipe where the uptake percentage is specified on a per-sub-recipe basis. In FIG. 2, the recipe for mixed greens salad with pickled onions, uses a sub-recipe called picked onions, which in turn uses a sub-recipe called pickling brine. Only a fraction of the pickling brine (about 6%) is actually absorbed by the pickled onions and hence makes its way into the mixed greens salad with pickled onions. Setting the uptake percentage to 6% for the pickling brine sub-recipe ensures that that only 6% of the nutrient content of the brine will be included in the nutrition facts calculated for the pickled onions and hence for the mixed greens salad with pickled onions.

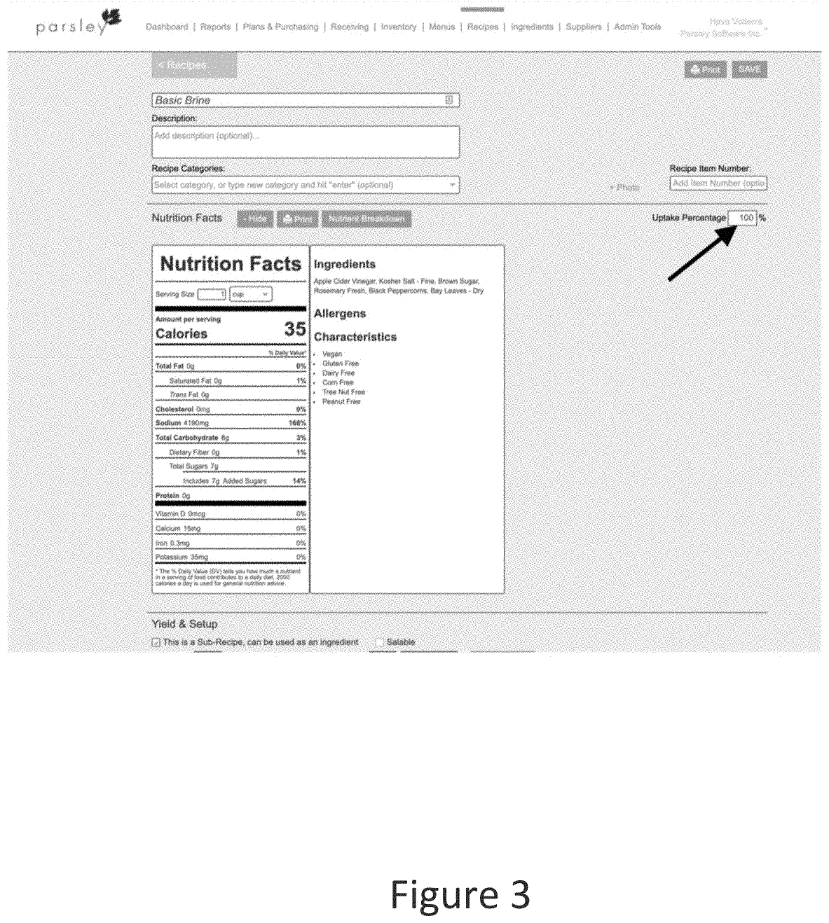

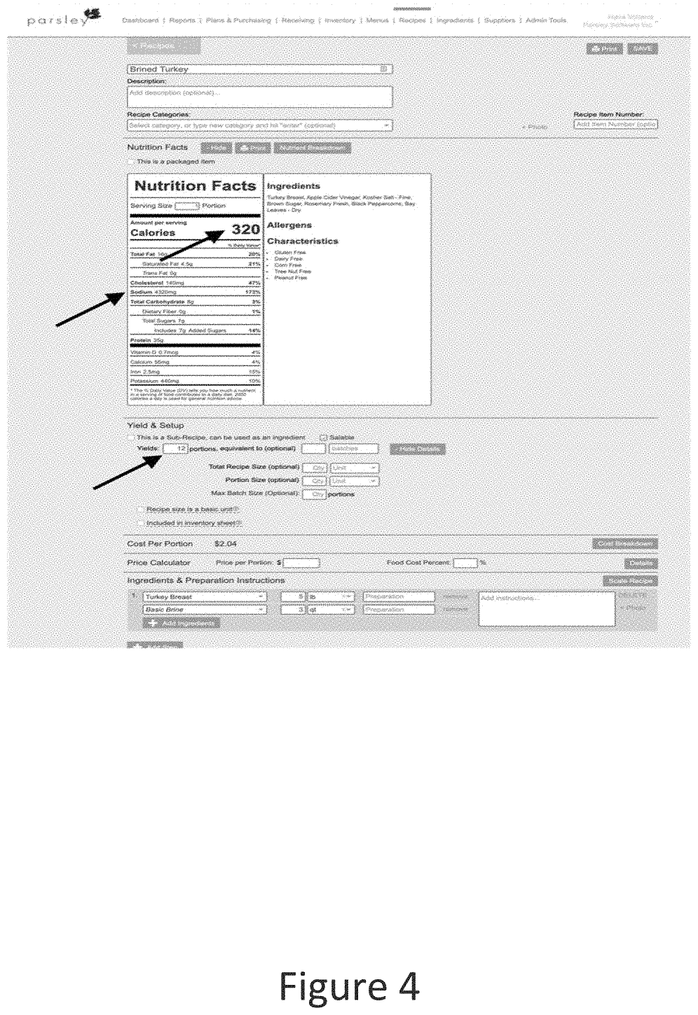

FIGS. 3-6 are computer screenshots showing an exemplary implementation of the uptake percentage on unified production food recipe management and nutrition content calculation software. We start with a basic brine (FIG. 3), with an Uptake Percentage set to 100%, and use it to brine turkey breast. This gives us 320 calories per portion (the recipe makes 12 portions), and 4320 mg of sodium (see FIG. 4) which is a very large quantity of sodium.

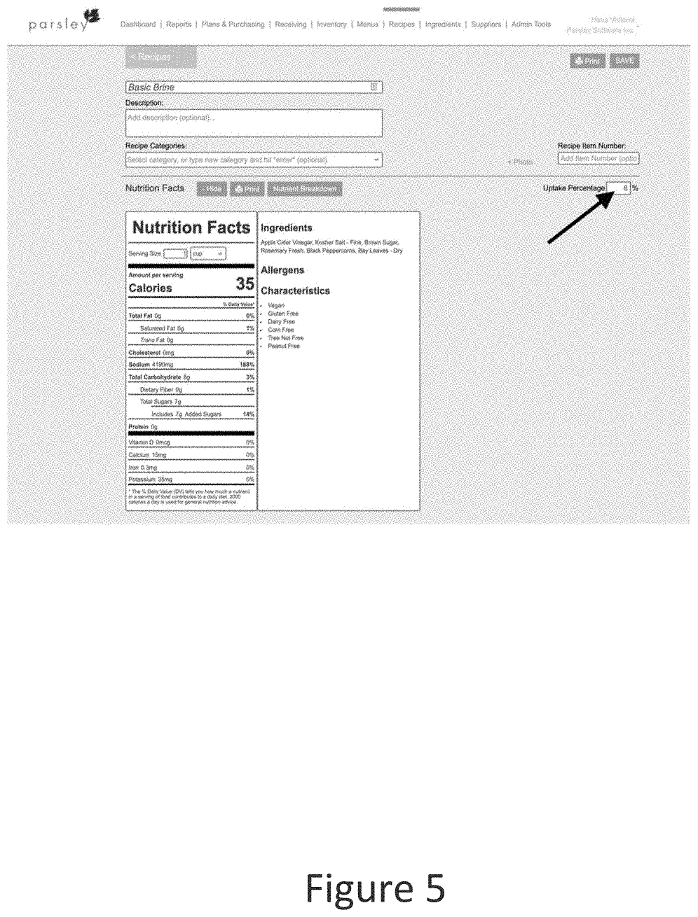

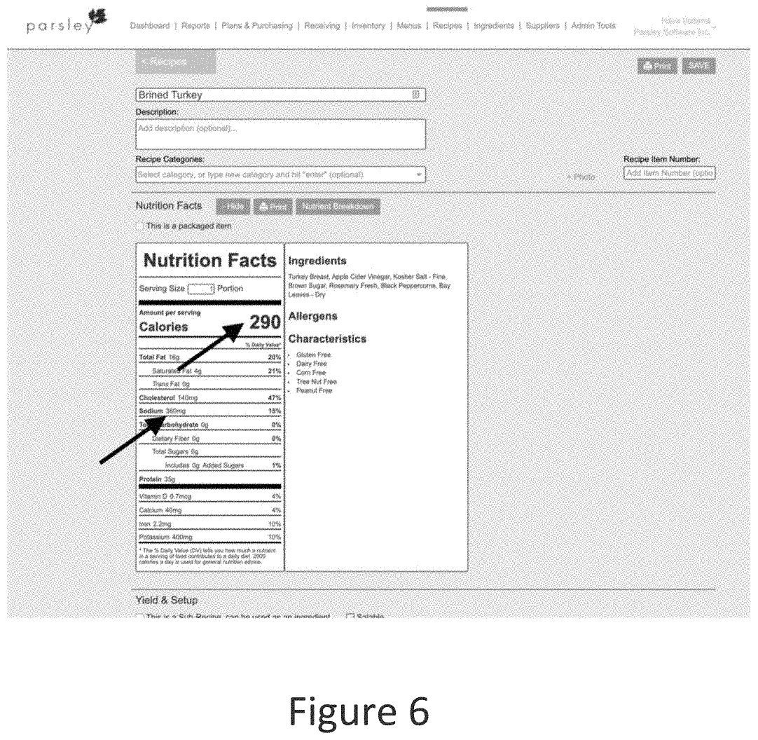

We then set the uptake percentage to a nominal value of 6% (FIG. 5). The amount of sodium in the brined turkey recipe goes down to 360 mg (FIG. 6). The calories go down too, although by much less, to 290 calories per portion. This is because most of the calories in this recipe come from the turkey breast, and only a small quantity come from the brown sugar that's in the brine. Still, that brown sugar is also reduced by the uptake percentage, since most of it remains in the discarded brine.

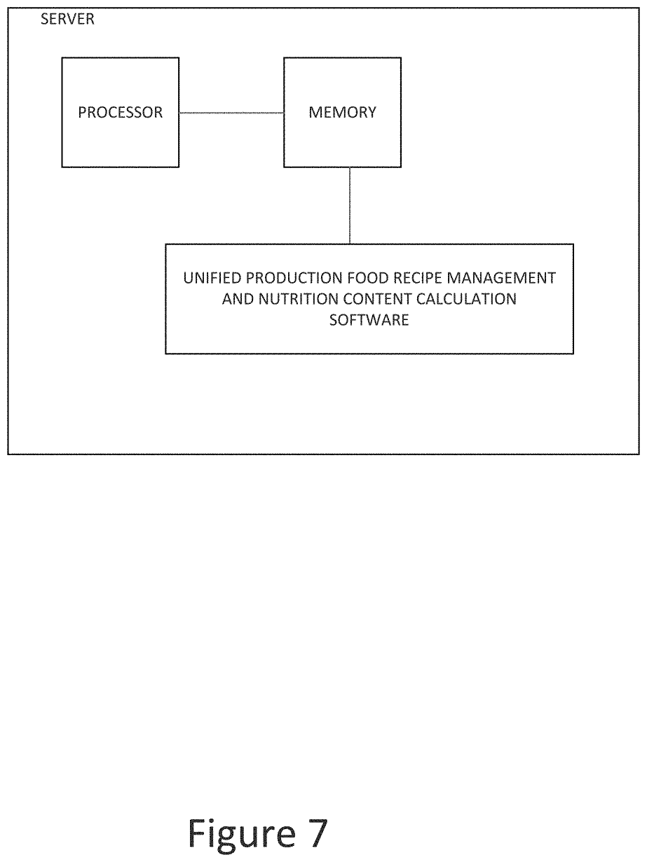

FIG. 7 is a block diagram illustrating a server configured to implement unified production food recipe management and nutrition calculation. In FIG. 7, the server includes at least one processor and a memory. Unified production food recipe management and nutrition calculation software may be stored in the memory and include computer executable instructions executable by the processor. The software may include a cloud-based or on premises web server, to which a user, such as a professional chef, connects using a web client, such as a web browser. The software then presents the user with screen shots, such as those illustrated in FIG. 3-6, that allow the user to define sub-recipes, specify uptake percentages on a per-sub-recipe basis, and calculate nutrition content using the production recipe ingredient quantities modified by the uptake percentages.

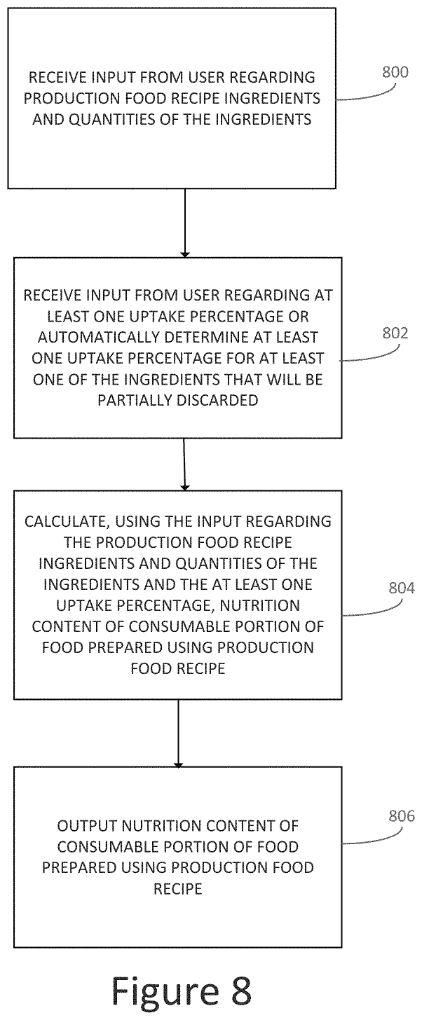

FIG. 8 is a flow chart illustrating an exemplary process for unified production food recipe management and nutrition content calculation. Referring to FIG. 8, in step 800, the process includes receiving input from a user regarding production food recipe ingredients and quantities of the ingredients used by the production food recipe. For example, the unified production food recipe management and nutrition content calculation software may present the user with a series of screens, such as those illustrated in FIG. 3-6, that allow the user the specify production food recipe ingredients, sub-recipes, and quantities. It should be noted that sub-recipes are optional, but placing ingredients in sub-recipes to which uptake percentages can be applied may be advantageous in that the sub-recipes can be re-used in other production food recipes, with the uptake percentages automatically applied, i.e., the user is not required to input the uptake percentage again when a sub-recipe for which an uptake percentage has been input or determined is used in another recipe.

In step 802, the process includes receiving input from the user regarding at least one uptake percentage or automatically determining at least one uptake percentage for at least one of the ingredients that will be partially discarded. For example, the unified production food recipe management and nutrition content software may automatically determine an uptake percentage based on one or more of the ingredients that will be partially discarded and present this value to the user. The one or more ingredients that will be partially discarded may be in a sub-recipe or outside of a sub-recipe. The uptake percentage may be determined using heuristics that are programmed into the software. The software may also allow the user to modify the automatically determined value, using the user interface illustrated in FIGS. 3-6.

In step 804, the process includes calculating, using the input regarding the production food recipe ingredients and quantities of the ingredients and the at least one uptake percentage, nutrition content of a consumable portion of food produced using the production recipe. For example, the unified production food recipe management and nutrition content calculation software may calculate the nutrition content of consumable portions of food prepared using the recipe using the ingredients, quantities, and uptake percentages input by the user.

In step 806, the process includes outputting the nutrition content of the consumable portion of food prepared using the production food recipe. For example, the production food recipe management and nutrition content calculation software may generate output, such as that illustrated in FIG. 6, which shows the nutrition content of a consumable portion of food prepared using the production food recipe.

It will be understood that various details of the subject matter described herein may be changed without departing from the scope of the subject matter described herein. Furthermore, the foregoing description is for the purpose of illustration only, and not for the purpose of limitation, as the subject matter described herein is defined by the claims as set forth hereinafter.

What is claimed is:

1. A method for unified production food recipe management and nutrition content calculation, the method comprising:
receiving input from a user regarding production food recipe ingredients and quantities of the ingredients used by a production food recipe;
receiving input from the user regarding at least one uptake percentage or automatically determining at least one uptake percentage for at least one of the ingredients that will be partially discarded;
calculating, using the input regarding the production food recipe ingredients and quantities of the ingredients and the at least one uptake percentage, nutrition content of a consumable portion of food prepared using the production food recipe; and.
outputting the nutrition content of the consumable portion of food prepared using the production food recipe.

2. The method of claim 1 wherein receiving input from the user regarding at least one uptake percentage or determining at least one uptake percentage includes automatically determining the at least one uptake percentage based on at least one ingredient type.

3. The method of claim 2 wherein receiving input from the user or determining at least one uptake percentage includes presenting the user with the at least one automatically determined uptake percentage and presenting the user with a graphical user interface element that allows the user to modify the at least one automatically determined uptake percentage.

4. The method of claim 1 wherein the at least one of the ingredients that will be partially discarded includes a brine, marinade, or cooking oil or fat.

5. The method of claim 1 comprising receiving input from the user regarding at least one sub-recipe and a quantity of the at least one sub-recipe to be used in the production food recipe and that includes the at least one of the ingredients that will be partially discarded and wherein receiving the input from the user or automatically determining the at least one uptake percentage includes receiving the input from the user or automatically determining the at least one uptake percentage for the at least one sub-recipe.

6. A system for unified production food recipe management and nutrition content calculation, the system comprising:
a server including at least one processor;
unified production food recipe management and nutrition calculation software implemented by the at least one processor for receiving input from a user regarding production food recipe ingredients, and quantities of the ingredients used by a production food recipe, receiving input from the user regarding at least one uptake percentage or automatically determining at least one uptake percentage for at least one of the ingredients that will be partially discarded, calculating, using the input regarding the production food recipe ingredients and quantities of the ingredients used by the production food recipe and the at least one uptake percentage, nutrition content of a consumable portion of food prepared using the production food recipe; and, and outputting the nutrition content of the consumable portion of food prepared using the production food recipe.

7. The system of claim 6 wherein receiving input from the user regarding at least one uptake percentage or determining at least one uptake percentage includes automatically determining the at least one uptake percentage based on at least one ingredient type.

8. The system of claim 6 wherein receiving input from the user or determining at least one uptake percentage includes presenting the user with the at least one automatically determined uptake percentage and presenting the user with a graphical user interface element that allows the user to modify the at least one automatically determined uptake percentage.

9. The system of claim 6 wherein the at least one of the ingredients that will be partially discarded includes a brine, marinade, or cooking oil or fat.

10. The system of claim 6 wherein the unified production food recipe management and nutrition calculation software is configured to receive input from the user regarding at least one sub-recipe and a quantity of the at least one sub-recipe to be used in the production food recipe and that includes the at least one of the ingredients that will be partially discarded and wherein receiving the input from the user or automatically determining the at least one uptake percentage includes receiving the input from the user or automatically determining the at least one uptake percentage for the at least one sub-recipe.

11. A non-transitory computer readable medium having stored thereon executable instructions that when executed by a processor of a computer control the computer to perform steps comprising:
receiving input from a user regarding production food recipe ingredients and quantities of the ingredients used by a production food recipe;
receiving input from the user regarding at least one uptake percentage or automatically determining at least one uptake percentage for at least one of the ingredients that will be partially discarded;
calculating, using the input regarding the production food recipe ingredients and quantities of the ingredients used by the production food recipe and the at least one uptake percentage, nutrition content of a consumable portion of food prepared using the production food recipe; and
outputting the nutrition content of the consumable portion of food prepared using the production food recipe.
